(19) United States
(12) Patent Application Publication
(10) Pub. No.: US 2016/0106593 A1
(43) Pub. Date: Apr. 21, 2016

(54) ADHESIVE BANDAGE WITH A GLITTERED OUTER SURFACE

(71) Applicant: Kitaru Innovations Inc., Lascelles (BB)

(72) Inventor: Kapoor Chandaria, Nairobi (KE)

(73) Assignee: Kitaru Innovations Inc.

(21) Appl. No.: 14/882,520

(22) Filed: Oct. 14, 2015

Related U.S. Application Data
(60) Provisional application No. 62/064,618, filed on Oct. 16, 2014.

(51) Int. Cl. A61F 13/00; A61F 13/02

(52) U.S. Cl. CPC A61F 13/00059; A61F 13/0206; A61F 13/022; A61F 13/0226; A61F 13/0289; A61F 2013/00153; A61F 2013/00289

(57) ABSTRACT

An adhesive bandage including a fabric layer having upper and lower surfaces; a first adhesive layer provided adjacent the fabric layer's lower surface; a gauze strip provided adjacent the first adhesive layer's lower surface; and a releasable covering applied over the gauze and possibly over any exposed regions of the first adhesive layer. A second adhesive layer may be provided adjacent the fabric layer's upper surface. A plurality of metallic glitter pieces may be provided adjacent an upper surface of the second adhesive layer or may be mixed into the second adhesive and the resultant mixture may be applied over the fabric layers' upper surface. A sealant layer may be applied over the glitter layer or the glitter/second adhesive mixture. The glitter layer may cover the entire upper surface of the bandage or be applied in discrete regions thereof. The glitter pieces may be arranged in decorative patterns or images.

### CROSS-REFERENCE TO RELATED APPLICATIONS

This application claims the benefit of U.S. Provisional Patent Application Ser. No. 62/064,618, filed Oct. 16, 2014, the entire disclosure of which is incorporated herein by reference.

### BACKGROUND OF THE INVENTION

1. Technical Field

This invention relates generally to medical supplies. More particularly the invention is directed to an adhesive bandage for placing over minor wounds. Specifically, the invention is directed to an adhesive bandage for children where the bandage has a non-skin contacting outer surface which includes a glittered finish.

2. Background Information

When younger children get hurt, such as by getting cut or scraping their knees, it may be necessary to apply an adhesive bandage over the wound. The adhesive bandage provides a shield over the wound and thereby may aid in preventing further injury or infection of the site. The adhesive bandage may also keep parts of the wounded tissue in closer proximity than would be the case if no bandage is used. This aids in promoting more rapid healing of the wound.

However, particularly with younger children, it may be very difficult to convince the child to allow someone to place an adhesive bandage over the wound. The child may relive the pain they have recently experienced incurring the injury and may be convinced that applying the adhesive bandage will add to that pain.

### SUMMARY

There is therefore a need in the art for an adhesive bandage that may tend to help the child turn their focus away from the wound and the pain involved therewith and thereby secure more ready cooperation from the child for applying the adhesive bandage over the injury.

The adhesive bandage disclosed herein includes a decorative outer surface which may aid in encouraging a child to allow the adhesive bandage to be placed over a wound. The adhesive bandage includes a glittered outer surface that is aesthetically pleasing to the eye. The glittered surface may include a shielding material that substantially prevents small pieces of glitter from breaking free from the adhesive bandage and accidentally entering the wound.

An adhesive bandage and a method of fabricating the same are disclosed. The bandage includes a fabric layer having an upper and lower surface; a first adhesive layer provided adjacent the fabric layer's lower surface; a gauze strip provided adjacent a portion of a first adhesive layer's lower; and one or more paper strips covering the gauze any exposed regions of the first adhesive layer. A second adhesive layer is provided adjacent the upper surface of the fabric layer. A plurality of glitter pieces may be provided adjacent an upper surface of the second adhesive layer or may be mixed into the second adhesive and the mixture then applied as a layer over the fabric layers' upper surface. A sealant layer may be applied over the glitter layer or glitter/second adhesive layer. The glitter layer may cover the entire upper surface of the bandage or be applied in discrete regions thereof. The glitter may form decorative patterns or images.

In one aspect, the invention may provide an adhesive bandage comprising a fabric layer having an upper surface and a lower surface; a first adhesive layer provided adjacent the lower surface of the fabric layer; a gauze strip provided adjacent a portion of a lower surface of the first adhesive layer; a second adhesive layer provided adjacent the upper surface of the fabric layer; and a plurality of glitter pieces being provided adjacent an upper surface of the second adhesive layer.

In another aspect, the invention may provide further comprise a sealant layer provided over the glitter pieces.

In another aspect, the invention may provide an adhesive bandage comprising a fabric layer having an upper surface and a lower surface; a first adhesive layer provided adjacent the lower surface of the fabric layer; a gauze strip provided adjacent a portion of a lower surface of the first adhesive layer; a covering releasably applied over at least the gauze strip; and a mixture of a second adhesive and a plurality of glitter pieces; wherein the mixture is provided adjacent the upper surface of the fabric layer.

In another aspect, the invention may provide a method of fabricating an adhesive bandage comprising the steps of providing a fabric strip having an upper surface and a lower surface; applying a first adhesive layer to the lower surface of the fabric strip; positioning a gauze strip adjacent a region of a lower surface of the first adhesive layer; adhering the gauze strip to the region of the lower surface of the first adhesive layer; and applying a glitter layer over the upper surface of the fabric strip.

In another aspect, the second adhesive layer is applied over the upper surface of the fabric strip; a plurality of glitter pieces is applied over an upper surface of the second adhesive layer and become adhered thereto.

In another aspect, a plurality of glitter pieces is mixed with a quantity of second adhesive to form a second adhesive/glitter mixture. The mixture is then applied over the upper surface of the fabric layer. A sealant layer may then be applied over any exposed regions of the upper surface of the fabric layer and the second adhesive/glitter mixture or only over the second adhesive/glitter mixture.

### BRIEF DESCRIPTION OF THE SEVERAL VIEWS OF THE DRAWINGS

A sample embodiment of the invention is set forth in the following description, is shown in the drawings and is particularly and distinctly pointed out and set forth in the appended claims.

FIG. 1 is a top view of an adhesive bandage in accordance with an aspect of the invention;

FIG. 2 is a bottom view of the bandage showing overlapping paper strips applied thereover;

FIG. 3 is a bottom view of the bandage with the paper strips removed therefrom;

FIG. 4A is a longitudinal cross-section of a first embodiment of the bandage taken along line 4A-4A of FIG. 1;

FIG. 4B is a longitudinal cross-section of a second embodiment of the bandage taken along line 4B-4B of FIG. 1;

FIG. 4C is a longitudinal cross-section of a third embodiment of the bandage taken along line 4C-4C of FIG. 1;

FIG. 5 is a top view of a fourth embodiment of the bandage showing the glitter applied in a decorative pattern at discrete intervals along the length of the bandage; and

FIG. 6 is a top view of a fifth embodiment of the bandage showing the glitter layer terminating a distance inwardly from a perimeter edge of the fabric layer.

Similar numbers refer to similar parts throughout the drawings.

### DETAILED DESCRIPTION

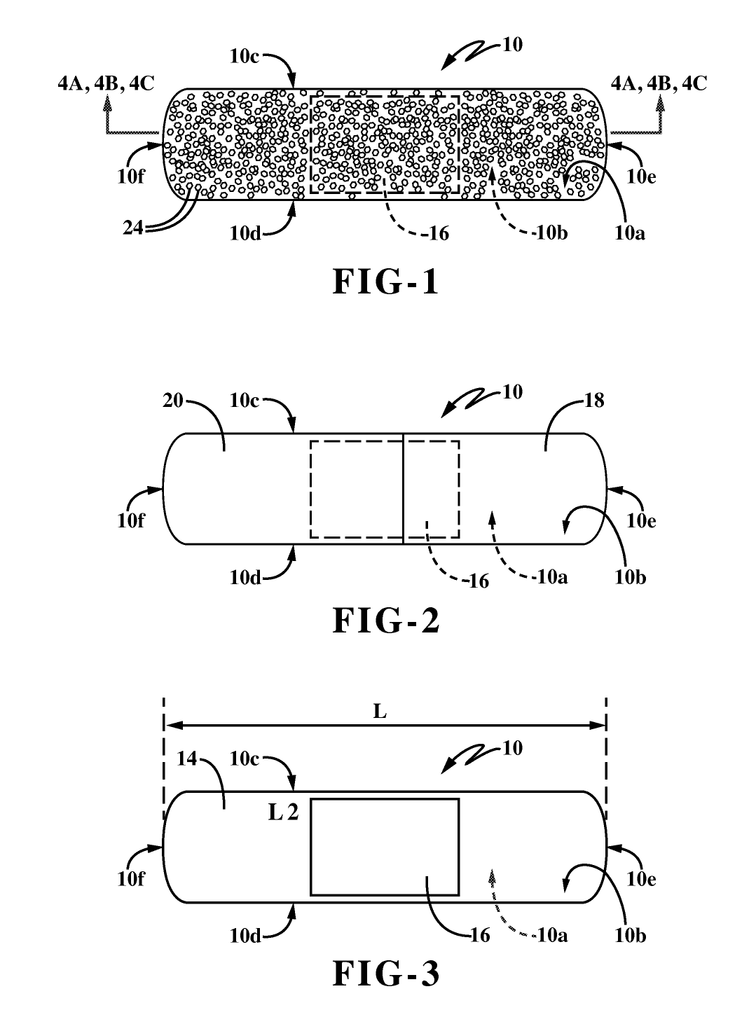

Referring to FIGS. 1-4A, there is shown an adhesive bandage in accordance with an aspect of the present invention, generally indicated at 10. The attached figures illustrate an elongate and generally rectangular bandage with rounded end regions. It will be understood, however, that bandage 10 may be fabricated in any of a number of different shapes, including but not limited to, square or circular.

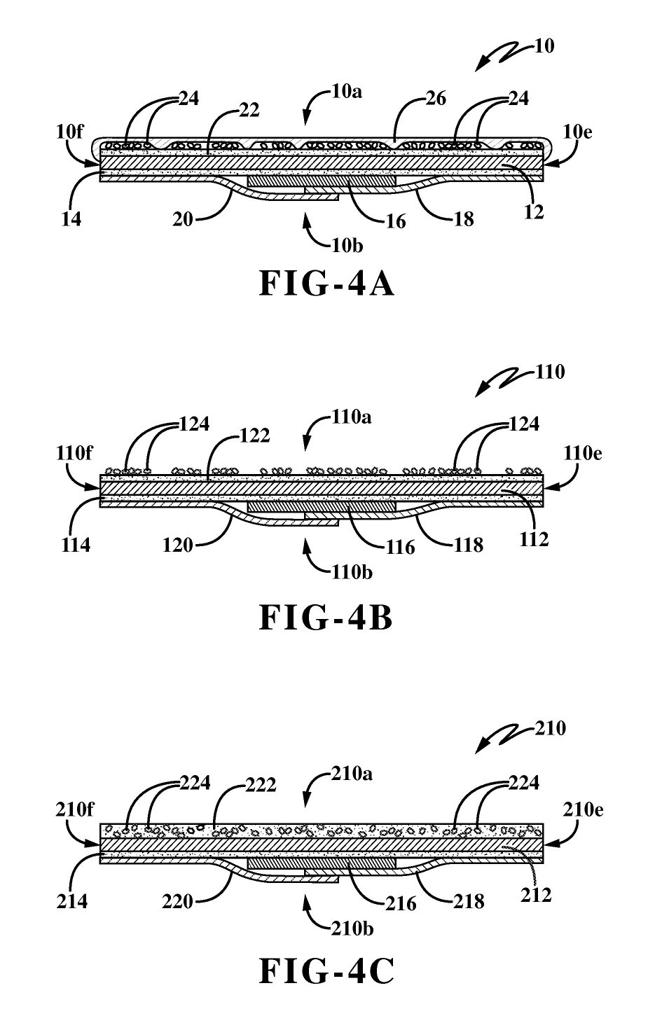

As illustrated, bandage 10 has an upper surface 10a, a lower surface 10b, a first side 10c, a second side 10d, a first end 10e, and a second end 10f. Bandage 10 may be comprised of several layers. A fabric layer 12 (FIG. 4A) may comprised of a flexible fabric such a stretchable elastic material or a plastic material. The flexible fabric may also be fabricated to be waterproof, water-resistant and/or breathable. The term “breathable” is directed to any fabric which permits air to flow therethrough to a greater or lesser extent so that the wound under the bandage 10 is able to dry out and heal. (It will be understood that fabric layer 12 may comprise a plurality of sub-layers that are positioned adjacent each other in order to provide the layer 12 with any desired properties, such as being waterproof or water-resistant. In these instances, the plurality of sub-layers may be considered to constitute the fabric layer 12.)

A first adhesive layer 14 may be provided adjacent a bottom surface of fabric layer 12. First adhesive layer 14 may cover substantially the entire bottom surface of fabric layer 12 or may be provided at discrete intervals in particular locations. A gauze strip 16 may be provided generally centrally on first adhesive layer 14 and may extend for a distance along the length “L” (FIG. 3) of adhesive layer 14. Gauze strip 16 will likely not cover substantially the entire first adhesive layer 14 because then none of the adhesive layer 14 would be positionable adjacent the user's skin to secure bandage 10 in place thereon.

Gauze strip 16 is the portion of bandage 10 that will be positioned over a wound. As such, gauze strip 16 needs to be kept as sterile as possible prior to application of bandage 10 on the user's skin. Bandage 10 is therefore provided with a releasable covering that is applied over gauze strip 16 and is removable therefrom when bandage 10 is to be used. The releasable covering may comprise one or more paper strips applied over gauze strip 16. For example, overlapping paper strips 18, 20 may be applied over the gauze strip 16 to keep the same sterile until use. Each paper strip 18, 20 may extend across the exposed regions of adhesive layer 14 and across gauze strip, with the paper strips extending from first to second side 10c, 10d of bandage 10. First paper strip 18 may extend from first end 10e of bandage 10 to slightly past the midway point of gauze strip 16. Second paper strip 20 may extend from second end 10f of bandage 10 to slightly past the midway point of gauze strip 16, and consequently overlaps a region of first paper strip 18 generally mid-way along the length “L” of bandage 10. It will be understood that other mechanisms for covering gauze strip 16 or keeping it sterile may be utilized in place of paper strips 18, 20.

In accordance with an aspect of a first embodiment of bandage 10, a second adhesive layer 22 (FIG. 4A) may be provided adjacent an upper surface of fabric layer 12. Second adhesive layer 22 may extend from proximate first end 10e of bandage 10 to proximate second end 10f thereof and from proximate first side 10c to proximate second side 10d.

Glitter 24 may be applied to an upper surface of second adhesive layer 22. Glitter 24 may comprise a plurality of small metallic pieces that adhere to second adhesive layer 22. The glitter 24 may be applied scattered around an in no particular pattern or may be applied in any desired decorative pattern and in any desired color or combination of colors. So, for example, glitter 24 may comprise a plurality of gold or silver metallic pieces that are applied so that the pieces cover substantially the entire upper surface of second adhesive layer 22. Alternatively, glitter 24 may be applied in one or more colors to form a decorative image such as a child's name or a graphic pattern or a favorite storybook or TV character. Glitter 24 may be applied in such a way that it becomes part of second adhesive layer 22 or glitter 24 may be applied so that it forms a layer that is positioned on top of second adhesive layer 22.

A clear sealant layer 26 may be provided over glitter 24. Sealant layer 26 may be provided in order to prevent any of the metallic glitter pieces from disengaging from the second adhesive layer 22 and accidentally dropping onto the child's skin. Any such stray glitter pieces may work their way into the wound and cause infection. Sealant layer 26 may extend outwardly beyond the perimeter edge of bandage 10 and may wrap around the perimeter edge of bandage 10 and extend at least as far down to the upper surface of fabric layer 12, terminating adjacent thereto. The sealant layer 26 may extend even further downwardly beyond the perimeter edge of fabric layer 12 and terminate adjacent a lower surface of first adhesive layer 14. FIG. 4A shows sealant layer 26 wrapping around each of the first and second ends 10e, 10f of bandage 10 and extending downwardly to terminate adjacent a middle of fabric layer 12. This placement helps ensure that no glitter pieces 24 escape from second adhesive layer 22.

FIG. 4B shows a second embodiment of the adhesive bandage, generally indicated at 110. Bandage 110 comprises a fabric layer 112, a first adhesive layer 114, a gauze strip 116, first and second paper strips 118, 120 and a second adhesive layer 122 that are substantially similar in structure and function to the fabric layer 12, first adhesive layer 14, gauze strip 16, paper strips 18, 20 and second adhesive layer 22. In this second embodiment, a plurality of glitter pieces are applied to the top surface of second adhesive layer 122 and adhere thereto to form glitter layer 124. No sealant layer is applied thereover.

FIG. 4C shows a third embodiment of the adhesive bandage, generally indicated at 210. Bandage 210 comprises a fabric layer 212, a first adhesive layer 214, a gauze strip 216, first and second paper strips 218, 220 that are substantially similar in structure and function to the fabric layer 12, first adhesive layer 14, gauze strip 16, and paper strips 18, 20. In this third embodiment, a plurality of glitter pieces is mixed into the second adhesive and the mixture of the glitter/second adhesive is provided in a layer 224 to the upper surface of fabric layer 212. The glitter pieces are thus trapped within the second adhesive and are therefore less prone to become disengaged from bandage 210.

It will be understood that instead of the second adhesive layer 22 being provided from a first end 10e to a second end 10f of bandage 10 and from a first side 10c to a second side 10d thereof, the second adhesive layer 22 may instead only be applied in a particular discrete decorative pattern on the upper surface of fabric layer 12. In this instance, the glitter will only be able to adhere to those specific discrete regions of the fabric layer 12 that have the second adhesive layer 22 applied thereto. The glitter and second adhesive layer may then form a raised pattern on the upper surface of the fabric layer 12.

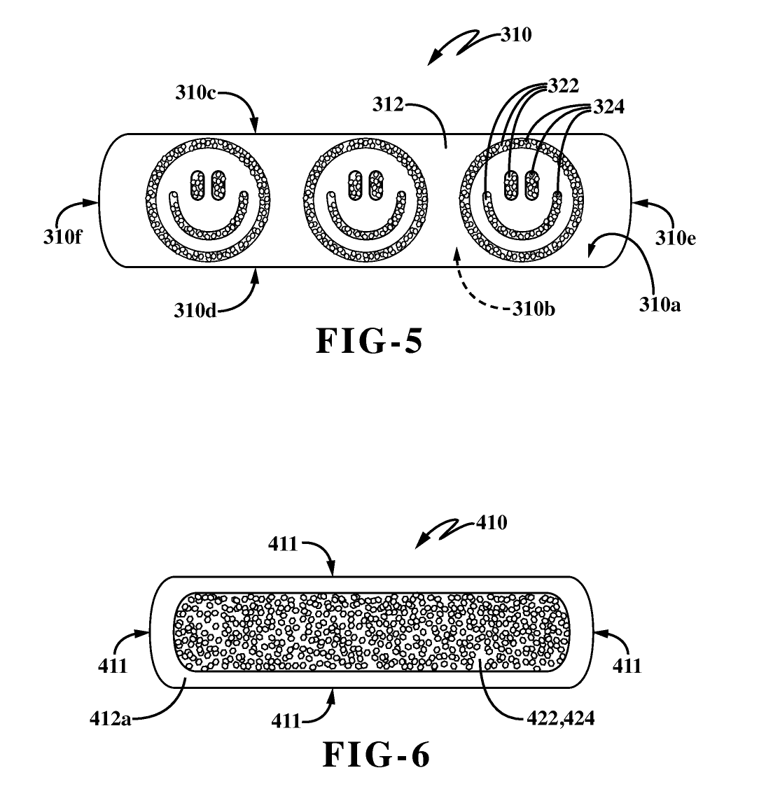

FIG. 5 shows a fourth embodiment of an adhesive bandage 310 showing the above-described fabrication of the bandage. Bandage 310 is constructed in a similar manner to bandage 210 but instead of the second adhesive/glitter mixture being applied over generally the entire upper surface of fabric layer 312, the second adhesive/glitter mixture 324 is only applied in a decorative pattern thereto. By way of example only, a number of “smiley-faces” 324 are provided at discrete intervals from each other along an upper surface of fabric layer 312. It will be understood that instead of a second adhesive/glitter mixture being used to form the “smiley-faces”, a second adhesive layer may be applied to the upper surface of fabric layer 312 in discrete locations only so as to form the general shape of the “smiley-face” and then glitter pieces 324 may be applied to the upper surface of fabric layer 312. The glitter pieces will only adhere to the second adhesive layer regions and not to the rest of the upper surface of the fabric layer 312. A sealant layer (not shown) may be applied over the exposed regions of the upper surface of the fabric layer 312 and over the second adhesive/glitter regions 324. As before, the sealant layer may aid in preventing glitter pieces from becoming dislodged from the regions 324. Alternatively, the sealant layer may only be applied over the smiley-face glitter regions 324.

It will be understood that the glitter may be provided in any of the manners described above, i.e., with a sealant layer thereover, or adhered to the top of the second adhesive layer or as an integral part of the second adhesive layer or in any other manner.

It will further be understood that more than one sealant layer may be applied over glitter layer 24, 124, 224 or glitter regions 324. So, for example, a first sealant layer may only be applied over glitter regions 324 and then a second sealant layer may be applied over the fabric layer 312 that is adjacent the glitter regions 324 and over the first sealant layer that is already applied to the glitter regions. The sealant may also be applied in a first layer that covers the fabric layer 312 and the glittered regions 324 and then a second sealant layer may be applied only over the sealed glittered regions 324 to make sure none of the glitter pieces escape therefrom.

It should further be understood that the second adhesive layer 22 may not be made of an adhesive, i.e., glue (as is the case of the first adhesive layer 14). Instead, the second adhesive layer 22 may be a layer of liquid plastic or a substance of similar nature that is able to adhere to fabric layer 12 and into which glitter pieces 24 may stick.

FIG. 6 shows a fifth embodiment of an adhesive bandage in accordance with the invention, generally indicated at 410. Bandage 410 may be substantially identical to bandage 10 with a second adhesive layer 422 provided adjacent an upper surface of fabric layer 412. FIG. 6 shows second adhesive layer 422 and glitter layer 424 starting a distance inwardly from a perimeter edge 411 of bandage 410. So, in other words, instead of extending all the way to the perimeter edge 411 of bandage 410, second adhesive layer 422 ends a short distance inwardly away from the perimeter edge 411 and therefore glitter layer 424 also terminates at the same distance inwardly from perimeter edge 411. A thin section of fabric layer 412 therefore forms a type of frame 412a around second adhesive layer 422 and glitter layer 424. A sealant layer (not shown) may be applied over the whole upper surface of bandage 410 and may even wrapper around and extend below perimeter edge 411.

A method of fabricating an adhesive bandage 10, 110, 210, or 310 is disclosed herein. The method will be described with reference to bandage 10 but applies to all of the other embodiments as well. The method includes providing a fabric strip 12 having an upper surface and a lower surface. A first adhesive layer 14 is applied to the lower surface of the fabric strip 12. A gauze strip 16 is positioned adjacent a region of a lower surface of the first adhesive layer 14 and is adhered thereto. One or more paper strips 18, 20 are applied over exposed regions of the first adhesive layer 14 and the gauze strip 16. A glitter layer is applied over the upper surface of the fabric strip 12 in a number of different ways. In a first embodiment, a second adhesive layer 22 is applied over the upper surface of the fabric strip; a plurality of glitter pieces 24 are applied over an upper surface of the second adhesive layer 22 and become adhered thereto; and a sealant layer 26 is applied over the plurality of adhered glitter pieces. The sealant layer 26 may be applied in such a way that the sealant layers extends downwardly beyond the edges of the second adhesive layer 22 and terminates proximate the fabric layer 12. The sealant layer 26 may be provided in such a way as to totally envelop fabric layer 12 and may be provided before first adhesive layer 14 and in such a way that sealant layer 26 covers the upper surface and lower surface of the fabric layer 12 and part of the sealant layer 26 is interposed between the lower surface of the fabric layer 12 and first adhesive layer 14. This latter configuration may be adopted to substantially prevent any glitter pieces 24 from accidentally becoming dislodged from glitter layer 24.

In a second embodiment; a second adhesive layer 122 is applied over the upper surface of the fabric strip 112; a plurality of glitter pieces 124 are applied over an upper surface of the second adhesive layer 122 and become adhered thereto.

In a third embodiment, a plurality of glitter pieces 224 is mixed with a quantity of adhesive to form a second adhesive/glitter mixture. The mixture is then applied over the upper surface of the fabric layer 212. A sealant layer may then be applied over any exposed regions of the upper surface of the fabric layer and the second adhesive/glitter mixture.

The glitter layer 24 may be provided in the form of a flexible sheet having metallic pieces embedded therein. In this instance, the flexible sheet with embedded glitter pieces may be adhered to second adhesive layer 22. Alternatively, the flexible sheet with embedded glitter pieces may, itself, be provided with an adhesive applied to a lower surface thereof. The flexible sheet may then be adhered to the upper surface of the fabric layer 12 by the adhesive.

In the foregoing description, certain terms have been used for brevity, clarity, and understanding. No unnecessary limitations are to be implied therefrom beyond the requirement of the prior art because such terms are used for descriptive purposes and are intended to be broadly construed.

Moreover, the description and illustration set out herein are an example and the invention is not limited to the exact details shown or described.

1. An adhesive bandage comprising:
a fabric layer having an upper surface and a lower surface;
a first adhesive layer provided adjacent the lower surface of the fabric layer;
a gauze strip provided adjacent a portion of a lower surface of the first adhesive layer;
a second adhesive layer provided adjacent the upper surface of the fabric layer; and
a plurality of glitter pieces being provided adjacent an upper surface of the second adhesive layer.

2. The adhesive bandage as defined in claim 1, further comprising a sealant layer provided over the plurality of glitter pieces.

3. The adhesive bandage as defined in claim 2, wherein the glitter pieces are not provided to a perimeter edge of the bandage but terminate a distance inwardly therefrom; and wherein the sealant layer extends outwardly beyond the glitter pieces and to the perimeter edge of the bandage.

4. The adhesive bandage as defined in claim 2, wherein the sealant layer extends from a first end of the bandage to a second end thereof; and from a first side of the bandage to a second side thereof.

5. The adhesive bandage as defined in claim 4, wherein the sealant layer extends over the first and second ends and the first and second sides of the bandage and the sealant layers terminates proximate one of the second adhesive layer or the fabric layer.

6. The adhesive bandage as defined in claim 1, wherein the glitter pieces form a decorative pattern.

7. The adhesive bandage as defined in claim 1, wherein the glitter pieces are of one or more colors.

8. The adhesive bandage as defined in claim 1, further comprising a releasable covering provided over the gauze strip.

9. An adhesive bandage comprising:
a fabric layer having an upper surface and a lower surface;
a first adhesive layer provided adjacent the lower surface of the fabric layer;
a gauze strip provided adjacent a portion of a lower surface of the first adhesive layer;
a covering releasably applied over at least the gauze strip; and
a mixture of a second adhesive and a plurality of glitter pieces; wherein the mixture is provided adjacent the upper surface of the fabric layer.

10. The adhesive bandage as defined in claim 9, further comprising a sealant layer provided over the mixture.

11. The adhesive bandage as defined in claim 10, wherein the sealant layer extends beyond a perimeter edge of the first fabric layer.

12. The adhesive bandage as defined in claim 9, wherein the mixture is provided on the upper surface of the fabric layer in a decorative pattern.

13. The adhesive bandage as defined in claim 12, further comprising a sealant layer, wherein the sealant layer is applied only over the mixture arranged in the decorative pattern.

14. The adhesive bandage as defined in claim 12, further comprising a sealant layer that extends to a perimeter edge of the fabric layer.

15. A method of fabricating an adhesive bandage comprising the steps of:
providing a fabric strip having an upper surface and a lower surface;
applying a first adhesive layer to the lower surface of the fabric strip;
positioning a gauze strip adjacent a region of a lower surface of the first adhesive layer;
adhering the gauze strip to the region of the lower surface of the first adhesive layer; and
applying a glitter layer over the upper surface of the fabric strip.

16. The method as defined in claim 15, wherein the step of applying the glitter layer includes:
applying a second adhesive layer to the upper surface of the fabric strip;
applying a plurality of glitter pieces to an upper surface of the second adhesive layer; and
adhering the plurality of glitter pieces to the fabric strip by way of the second adhesive layer.

17. The method as defined in claim 15, wherein the step of applying the glitter layer includes:
mixing a plurality of glitter pieces with a second adhesive to form a second adhesive/glitter mixture; and
applying the second adhesive/glitter mixture to the upper surface of the fabric strip.

18. The method as defined in claim 15, further comprising the step of applying a sealant layer over the glitter layer.

19. The method as defined in claim 15, wherein the step of applying the glitter layer includes:
providing a second adhesive layer in discrete locations on the upper surface of the fabric strip; and
placing a plurality of glitter pieces in a decorative pattern on the second adhesive layer.

20. The method as defined in claim 19, further comprising the step of applying a sealant layer over the glitter layer; and wherein the sealant layer is applied only over the plurality of glitter pieces or over the fabric strip and the plurality of glitter pieces.
